(19) United States
(12) Patent Application Publication
(10) Pub. No.: US 2025/0195635 A1
(43) Pub. Date: Jun. 19, 2025

(54) SALMONELLA FREE EGG PRODUCTION METHODS AND SYSTEMS

(71) Applicant: LUBERSKI, INC. DBA HIDDEN VILLA RANCH, Fullerton, CA (US)

(72) Inventor: Timothy LUBERSKI, Yorba Linda, CA (US)

(73) Assignee: LUBERSKI, INC. DBA HIDDEN VILLA RANCH, Fullerton, CA (US)

(21) Appl. No.: 18/843,692

(22) PCT Filed: Mar. 3, 2023
(86) PCT No.: PCT/US2023/014472

§ 371 Date: Sep. 3, 2024

Related U.S. Application Data
(60) Provisional application No. 63/316,375, filed on Mar. 3, 2022.

(51) Int. Cl. A61K 39/112; A01K 45/00; A61K 39/00; A61K 39/02; A61K 39/13; A61K 39/205; A61K 39/29

(52) U.S. Cl. CPC A61K 39/0275; A61K 39/099; A61K 39/13; A61K 39/205; A61K 39/29; A01K 45/00; A61K 2039/52; A61K 2039/5252; A61K 2039/552

(57) ABSTRACT

A method of generating a Salmonella free egg is disclosed. The method includes administering a vaccination to a poultry animal that is less than one year old. The method further includes boosting the vaccination of the poultry animal when the poultry animal is between about 50 weeks old and about 60 weeks old, the boosting comprising administering killed virus if the poultry animal molted subsequent to the vaccination, and the boosting comprising administering live virus if the poultry animal did not molt subsequent to the vaccination at the time of boosting.

### CROSS REFERENCE TO PRIOR APPLICATION

This application is a National Stage application of the International Application No. PCT/US23/14472, entitled as “SALMONELLA FREE EGG PRODUCTION METHODS AND SYSTEMS”, filed Mar. 3, 2023, which claims the benefit of U.S. Provisional Patent Application No. 63/316,375, entitled as “SALMONELLA FREE EGG PRODUCTION METHODS AND SYSTEMS”, filed Mar. 3, 2022, which are incorporated by reference in their entirety.

### FIELD OF THE INVENTION

This disclosure relates to the field of creating Salmonella free eggs or significantly reducing Salmonella inside and outside eggs. In particular, this disclosure relates to treating animals through a vaccination protocol that eliminates the growth of Salmonella Enteritidis.

### BACKGROUND

Most types of Salmonella live in the intestinal tracts of animals and birds and are transmitted to humans by contaminated foods of animal origin. Stringent procedures for cleaning and inspecting eggs were implemented and have made salmonellosis caused by external fecal contamination of eggshells rare. However, unlike egg borne salmonellosis of past decades, the current epidemic is due to intact and disinfected grade A eggs. The reason for this is that Salmonella enteritidis silently infects the ovaries of healthy appearing hens and contaminates the eggs before the shells are formed.

Healthy adults and children are at risk for egg-associated salmonellosis, but the elderly, infants, and persons with impaired immune systems are at increased risk for serious illness. In these persons, a relatively small number of Salmonella bacteria can cause severe illness. Most of the deaths caused by Salmonella Enteritidis have occurred among the elderly in nursing homes. Egg-containing dishes prepared for any of these high-risk persons in hospitals, in nursing homes, in restaurants, or at home should be thoroughly cooked and served promptly.

Accordingly, a better process of generating a Salmonella free egg is needed to help avoid risk to the elderly and the immune compromised. Moreover, a Salmonella free egg is needed for everyone. Vaccines for attenuating Salmonella are disclosed for example, in U.S. Pat. Nos. 6,399,074, 8,889,121, 8,133,493, each of which are incorporated herein by reference in their entirety.

### SUMMARY

The present disclosure discloses a method for generating a Salmonella free egg. The method includes administering a vaccination to a poultry animal that is less than one year old. In various embodiments, the vaccinated poultry animal is less than one year old and has gone through a California vaccination protocol. The method further includes boosting the vaccination of the poultry animal when the poultry animal is between about 50 weeks old and about 60 weeks old, the boosting comprising administering killed virus if the poultry animal molted subsequent to the vaccination, and the boosting comprising administering live virus if the poultry animal did not molt subsequent to the vaccination. In accordance with the aspects of the disclosure, the killed virus may include at least one of an inactivated poliovirus (IPV) vaccine, a whole cell pertussis vaccine, a rabies vaccine, a hepatitis A virus vaccine, and a combination thereof. In accordance with the aspects of the disclosure, the live virus may include at least one of a varicella-zoster vaccine, oral poliovirus (OPV) vaccine, a yellow fever virus vaccine, and a combination thereof. In accordance with the aspects of the disclosure, the method may further include implementing a Salmonella Enteritidis environmental monitoring program, which includes at least one testing protocol. In accordance with the aspects of the disclosure, the testing protocol may include testing of chick papers at delivery. In accordance with the aspects of the disclosure, the testing protocol may include performing an environmental test at about 14 weeks to about 16 weeks post molt. In accordance with the aspects of the disclosure, the testing protocol may include performing an environmental test at about 40 weeks to about 45 weeks post molt. In accordance with the aspects of the disclosure, the testing protocol may include performing an environmental test at about 4 weeks to about 6 weeks post molt.

Another general aspect is a system for generating a Salmonella free egg. The system includes a poultry animal that was vaccinated when it was less than one year old. The poultry animal was boosted when the poultry animal was between about 50 weeks old and about 60 weeks old, the boosting comprising administering killed virus if the poultry animal molted subsequent to the vaccination, and the boosting comprising administering live virus if the poultry animal did not molt subsequent to the vaccination. The killed virus may include at least 1 of an inactivated poliovirus (IPV) vaccine, a whole cell pertussis vaccine, a rabies vaccine, hepatitis A virus vaccine, and a combination thereof. The live virus may include at least 1 of a varicella-zoster vaccine, oral poliovirus (OPV) vaccine, a yellow fever virus vaccine, and a combination thereof. The poultry animal may be monitored by a Salmonella enteritidis environmental monitoring program, which includes at least one testing protocol. The testing protocol may include testing of chick papers at delivery. The testing protocol may include performing an environmental test at 14 weeks to about 16 weeks post molt. The testing protocol may include performing an environmental test at about 40 weeks to about 45 weeks post molt.

An exemplary embodiment is a method of generating a Salmonella free egg. The method includes administering a vaccination to a poultry animal that is less than 1-year-old. The method includes boosting the vaccination to the poultry animal when the poultry animal is between about 50 weeks old and about 60 weeks old, the boosting comprising administering killed virus if the poultry animal molted subsequent to the vaccination, and the boosting comprising administering live virus if the poultry animal did not molt subsequent to the vaccination. The administering live virus includes providing live virus prior to molting and providing live virus again subsequent to molting The method includes harvesting an egg that was laid by the poultry animal. The killed virus may include at least 1 of an inactivated poliovirus (IPV) vaccine, a whole cell pertussis vaccine, a rabies vaccine, hepatitis A vaccine, and a combination thereof. The live virus may include at least 1 of a varicella-zoster vaccine, oral poliovirus (OPV) vaccine, a yellow fever virus vaccine, and a combination thereof. The method may further include implementing a Salmonella enteritidis environmental monitoring program, which includes at least one testing protocol.

### BRIEF DESCRIPTION OF THE DRAWINGS

FIG. 1 illustrates a sample diagram of an infection cycle in a chicken that lays eggs.

FIG. 2 illustrates a vaccination schedule for a chicken in an embodiment of the disclosed subject matter.

FIG. 3 illustrates an environmental testing schedule for a chicken in an embodiment of the disclosed subject matter.

FIG. 4 is a flow diagram of a method of generating a Salmonella free egg, according to an embodiment of the present disclosure.

### DETAILED DESCRIPTION

Embodiments, of the present disclosure, will now be described with reference to the accompanying drawings.

Embodiments are provided so as to convey the scope of the present disclosure thoroughly and fully to the person skilled in the art. Numerous details, are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The disclosed subject matter is a system and method for generating and collecting eggs from chickens that are free of Salmonella infection. Besides chickens, the disclosed subject matter may be practiced with other forms of poultry or agricultural birds including but not limited to turkeys, ducks, geese, pheasants, guinea fowls, quails, squabs, and ostriches. Embodiments herein with references to any type of agricultural bird are not intended to be limited to a single species, but instead may be applicable to many or all agricultural birds.

In an exemplary embodiment, a chicken is vaccinated when it is less than one year old. The chicken may be vaccinated by various protocols such as the California vaccination protocol. Then, when the chicken is around 55 weeks old, the chicken is vaccinated again or boosted. The term “boosted”, as used herein, refers to a 2nd vaccination of the chicken where the 2nd vaccination comprises substantially the same antigens of the 1st vaccination. The boosting is modified based on whether the chicken molted subsequent to the 1st vaccination. Molting is a process where chickens shed and regrow their feathers. Molting tends to occur seasonally and often occurs during late Summer or the Fall. Accordingly, the chicken molts not based on its age but based on the time of year. Molting in birds refers to the process of shedding and replacing old feathers with new ones. Birds molt regularly to replace feathers that have become worn, damaged, or faded, or to grow new feathers for breeding or migration. During molting, birds typically lose their old feathers in a sequential pattern, with feathers on different parts of the body being replaced at different times. The timing and duration of molting can vary depending on the species, age, and environmental conditions. Molting can be a physically demanding process for birds, as it requires a lot of energy and nutrients to grow new feathers. During this time, birds may also be more vulnerable to predators and may alter their behavior and migration patterns. Overall, molting is an important part of a bird's life cycle, as it enables them to maintain its flight, insulation, and communication abilities, and helps them adapt to changing environmental conditions. Molting can lower the immunization response of the bird from vaccines that were delivered earlier.

As stated above, the chicken is boosted at or around 55 weeks of age. Since molting is a seasonal process and not dependent on age alone, some chickens will molt before they are boosted and some chickens will not. If the chicken is boosted before the chicken molts, the chicken is administered live virus before molting and live virus again after the chicken molts. On the other hand, if the chicken is boosted after the chicken molts, the chicken is administered killed virus. Eggs are then collected from the boosted chicken.

It is believed that the disclosed process for boosting the chicken results in lower probability of Salmonella infection in eggs laid by the chicken when compared to contemporary farming practices. Accordingly, eggs produced by the disclosed process are safer for human consumption.

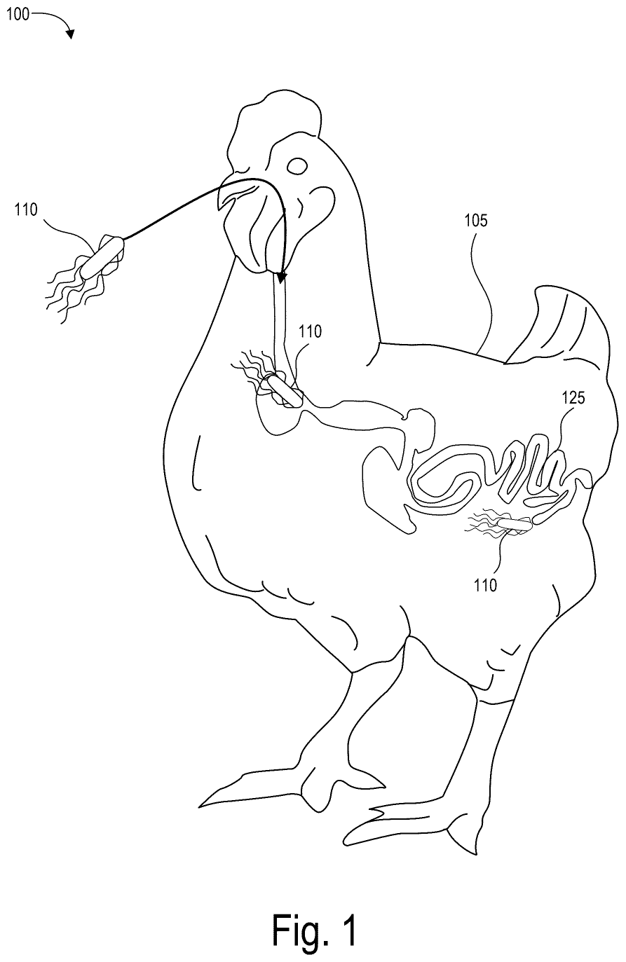

Referring to FIG. 1, FIG. 1 illustrates a sample diagram 100 of an infection cycle. Salmonella 110 can be transmitted to humans through consumption of contaminated products and poultry through horizontal/vertical transmission. The Salmonella 110 may be orally passed into the chicken 105 and survives an acid pH and reaches the small intestine 125. The pH of the small intestine 125 gradually increases from 6-7.5. The Salmonella 110 is then transported through the mucosa of the chicken via M-cells in order to gain access to Peyer's patches. The Salmonella is up taken and processed by dendritic cells for antigen presentation. The Salmonella is further phagocytosed by macrophages with the intent to eliminate pathogens. Pathogens are organisms that may tend to cause diseases.

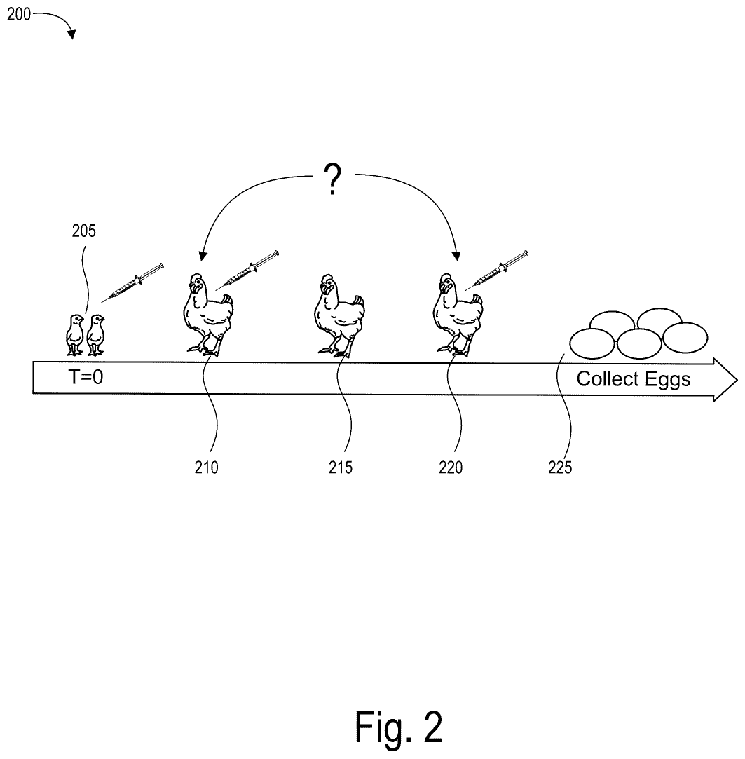

Referring to FIG. 2, FIG. 2 illustrates a vaccination schedule 200 for a chicken in an embodiment of the disclosed subject matter. The vaccination schedule 200 comprises vaccinating a chick 205 after it hatches. The vaccination may occur at various times according to various farming practices. In all cases, the vaccination will occur before the chicken is 1-year-old. During a molting season, which is typically around late Summer or Fall, the chicken 215 will shed its feathers and regrow them. During this time the chicken will typically stop laying eggs. The age of the chicken when it molts is dependent on the time of year that it hatches and will vary from chicken to chicken. Accordingly, some chickens will molt before they are 55 weeks old and some will molt after they are 55 weeks old.

During molt, the chickens stop laying eggs and built up their nutrition reserves, due to which it is essential that the chickens have a high-quality diet. Feeding a high nutrition diet during molt will help the chickens get back to laying eggs as quickly as possible. During the molting process, chickens may also lose feathers, which are grown back within a time period of about 3 weeks to 4 weeks.

A chicken 220 that molts before it is 55 weeks old is boosted differently from a chicken 210 that molts after it is 55 weeks old. The chicken 210 that ages 55 weeks before molting is boosted through administration of live virus at 55 weeks old. The chicken 210 is then administered live virus again after it molts. On the other hand, a chicken 220 that molts before it ages 55 weeks is administered killed virus at 55 weeks old with no further administrations.

Eggs 225, which are laid by the chicken, are then collected for human consumption subsequent to boosting the chicken. The boosting lowers the risk of Salmonella infection in the chicken, which lowers the risk of Salmonella infection in any eggs that it lays. In various embodiments, the chicken is boosted at about 55 weeks of age. In various embodiments, the chicken is boosted at between about 54 weeks of age and about 56 weeks of age. In various embodiments, the chicken is boosted at about 56 weeks of age. In various embodiments, the chicken is boosted at between about 53 weeks of age and about 57 weeks of age. In various embodiments, the chicken is boosted at between about 52 weeks of age and about 58 weeks of age. In various embodiments, the chicken is boosted at between about 51 weeks of age and about 59 weeks of age. In various embodiments, the chicken is boosted at between about 50 weeks of age and about 60 weeks of age. In various embodiments, the chicken is boosted at between about 49 weeks of age and about 61 weeks of age. In various embodiments, the chicken is boosted at between about 48 weeks of age and about 62 weeks of age.

The timing for the term boosting, as used herein, refers to the start of boosting for chickens that are boosted before molting and require an administration at the time of boosting and a 2nd administration after the chicken molts. Accordingly, the time of boosting refers to the 1st administration for those chickens. The 2nd administration may be administered at about 4 weeks after the chicken molts. In various embodiments, the 2nd administration may be between about 3 weeks and about 5 weeks after the chicken molts. In various embodiments, the 2nd administration may be between about 3 weeks and about 6 weeks after the chicken molts. In various embodiments, the 2nd administration may be between about 2 weeks and about 6 weeks after the chicken molts. In various embodiments, the 2nd administration may be between about 4 weeks and about 8 weeks after the chicken molts. In various embodiments, the 2nd ministration may be between about 6 weeks and about 10 weeks after the chicken molts. In various embodiments, the 2nd administration may be between about 8 weeks and about 12 weeks after the chicken molts.

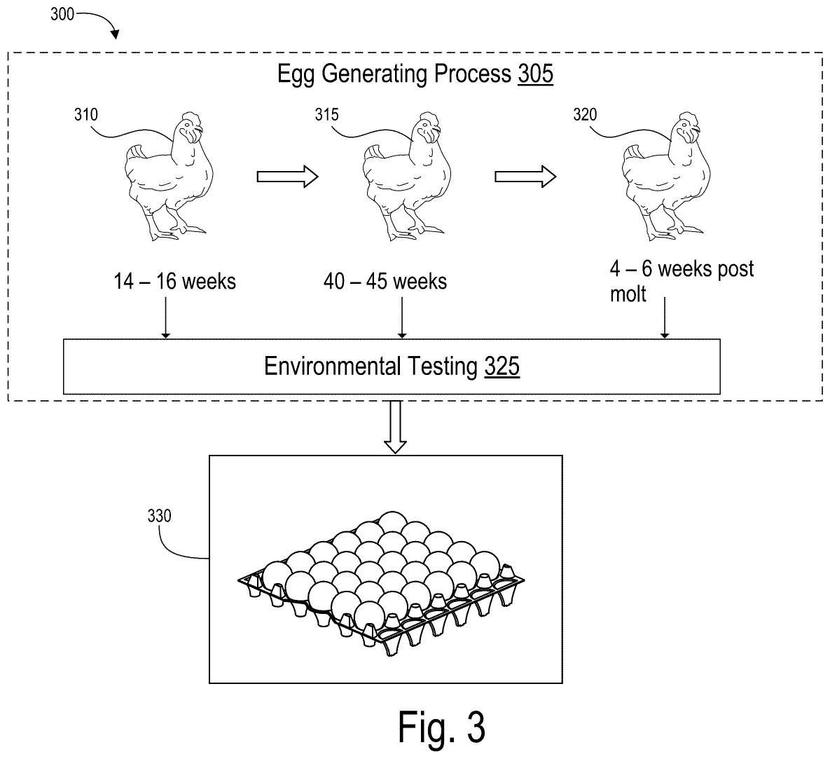

Referring to FIG. 3, FIG. 3 illustrates an environmental testing schedule 300 for a chicken 310 in an embodiment of the disclosed subject matter. Implementing a Salmonella enteritidis environmental monitoring program 305 includes the following testing protocols. The testing protocols includes i) testing of chick papers at delivery, (ii) performing an Environmental Test 310 at 14-16 weeks, (iii) performing an Environmental Test 315 at 40-45 weeks, and (iv) performing an Environmental Test 320 at 4-6 weeks post molt, and (v) Environmental Test pre depopulation.

Chick papers are special types of papers that are developed for the poultry industry. Chick papers have high rusting properties and are generally used as a feeding area for the chickens. Chick papers come in ideal sizes, for example 140×100×97 cm, which gives an ideal spreading for each chicken, due to which possibilities of spillage of feed is reduced. Chick papers are also highly degradable, due to which it is not necessary to remove the chick paper from the poultry house as it degrades in about 3 days to 5 days.

An Environmental Test may be performed post the molting process. The environmental test measures the performance of the Salmonella enteritidis environmental monitoring program under specified environmental conditions. For example, the specified environmental conditions may be high/low temperatures, swift variations in temperatures, radiations, acoustic measures, and the like. The environmental test helps in exposing weaknesses in the Salmonella enteritidis environmental monitoring program or performance at certain levels of the Salmonella enteritidis environmental monitoring program. Eggs 330 produced from the disclosed Salmonella enteritidis environmental monitoring program 305 are harvested from boosted chickens. The eggs are believed to have a lower probability of contamination than eggs produced in contemporary farming practices.

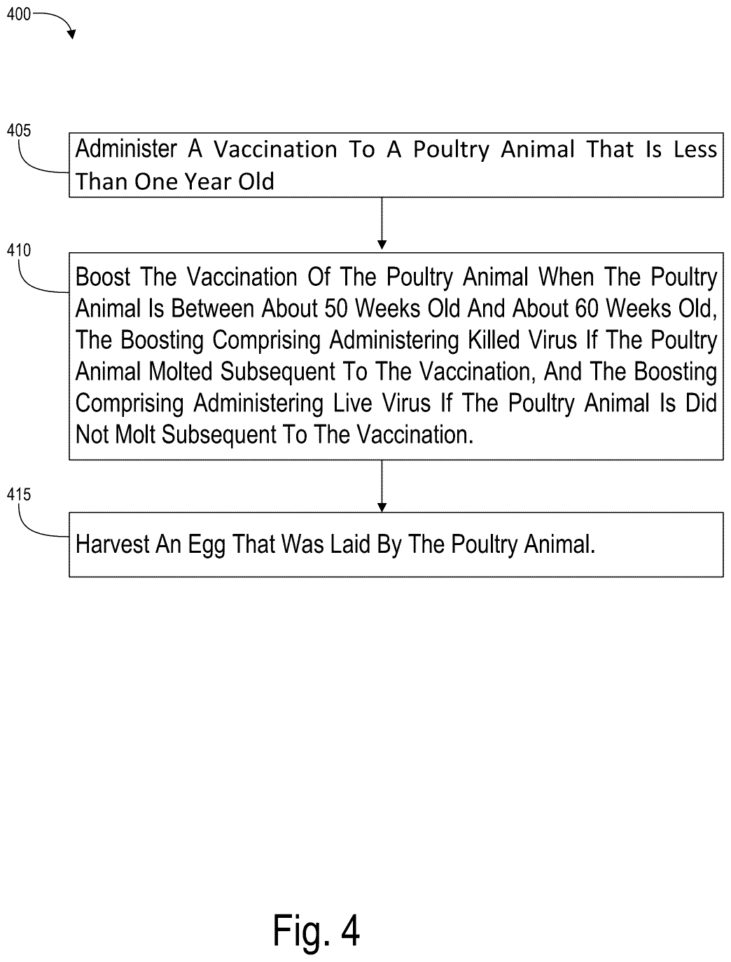

Referring to FIG. 4, FIG. 4 illustrates a flow chart of a process 400 of generating a Salmonella free egg, according to an embodiment of the present disclosure. At step 405, the process administers a vaccination to a poultry animal that is less than 1 year old. In various embodiments the 1st vaccination will occur when the chick is a few days old.

At step 410, the process may boost the vaccination to the poultry animal when the poultry animal is between about 50 weeks old and about 60 weeks old, the boost comprising administering killed virus if the poultry animal molted subsequent to the vaccination at the time of boosting, and the boosting comprising administering live virus if the poultry animal did not molt subsequent to the vaccination at the time of boosting. Because the poultry animal may age to between about 50 weeks of age and about 60 weeks of age before or after it molts, various poultry animals will be treated differently under the disclosed process based on the time that they molt. The time that the poultry animal reaches 55 weeks of age+about 7 weeks is referred to herein as the time of boosting. A poultry animal that reaches the time of boosting before it molts is administered live virus for boosting and a poultry animal that reaches the time of boosting after it molts is administered killed virus.

At step 415, the process may harvest an egg that was laid by the poultry animal. It is anticipated that the egg will be made ready for human consumption. In various embodiments, one or more eggs that are harvested by the poultry animal may be tested for various pathogens.

In an embodiment, the killed virus includes at least one of an inactivated poliovirus (IPV) vaccine, a whole cell pertussis vaccine, a rabies vaccine, a hepatitis A virus vaccine, and a combination thereof. The killed virus does not revert to its virulent form after being injected. This prevents multiplication after administration, due to which the killed virus does not spread to the environment and to humans. The killed virus also has no danger of vaccine contamination. However, the killed virus may elicit a shorter length of protection, due to which they are more likely to require boosters to create long term immunity. Also, adjuvants in killed vaccines may also be required at some times. Adjuvants are ingredients used in helping to create a stronger immune response.

In an embodiment, the live virus includes at least one of a varicella-zoster vaccine, oral poliovirus (OPV) vaccine, a yellow fever virus vaccine, and a combination thereof. The live virus may elicit both a cell-mediated and humoral immune responses, due to which they may rarely require a booster. The live virus is also not administered intramuscularly. This leads to an increase in a quality of the tissue, due to which value of the live virus increases. Further, the live virus does not often require adjuvants as much as the killed virus, due to which the immunity of the live virus is better that the immunity of the killed virus. However, at some times, the live virus may interfere with Salmonella monitoring programs, due to which the live virus may get contaminated at some instances.

Further, apart from the above-listed vaccines, the currently available vaccines that may be used, is shown in the table below:

| | | | | Administrative |
|---|---|---|---|---|
| Company/Vaccine | Live | Killed | Bird | Route |
| Zoetis/POULVAC ® ST | X | | Boilers/Layers | Spray |
| Zoetis/POULVAC ® Se | | X | Boilers/Layers | Injection |
| Zoetis/POULVAC ® SE-ND-IB | | X | Boilers/Layers | Injection |
| IDT Bio/SALMOVAC ® SE | X | | Boilers/Layers | Oral |
| IDT Bio/ZOOSALORAL H | X | | Breeders/Layers | Oral |
| CEVA/LAYERMUNE ® SE | | X | Breeders/Layers | Injection |
| CEVA/LAYERMUNE ® RANGE | | X | Breeders/Layers | Injection |
| ELANCO/AviPro ® Megan ® Vac 1 | X | | Young Chickens | Spray |
| ELANCO/AviPro ® Megan ® Egg | X | | Layers/turkeys | Spray |
| ELANCO/AviPro ® 329 ND-IB2- | | | | |
| SE4 | | X | Breeders/Layers | Injection |

Young Chickens are defined as “one day of age”. In an embodiment, the method may include maintaining a flock health by vaccinations and monitoring and periodic necropsy of mortality or cull birds. All chickens are vaccinated using a vaccination program to protect against infection with Salmonella enteritidis (SE), which includes a killed or inactivated vaccine, or a demonstrated equivalent SE vaccination program as determined by a licensed veterinarian.

The method described herein duly selects a chicken from vaccinated chickens at the time of boosting and accordingly injects the chicken with a killed virus or a live virus. The killed virus does not return to its virulent form after being injected. Thus, possibilities of infection multiplication after injection are reduced. This thereby prevents the virus from spreading to the environment and humans, especially the elderly. Further, the live virus is not required to be administered intramuscularly after injection. This also prevents spreading of the virus. Thus, the method described herein discloses an improved process for generating a Salmonella free egg, due to which infants and persons with impaired immune systems are at a lesser risk for egg-associated salmonellosis.

The foregoing description of the embodiments has been provided for purposes of illustration and not intended to limit the scope of the present disclosure. Individual components of a particular embodiment are generally not limited to that particular embodiment but are interchangeable. Such variations are not to be regarded as a departure from the present disclosure, and all such modifications are considered to be within the scope of the present disclosure.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A method of generating a Salmonella free egg, the method comprising:
administering a vaccination to a poultry animal that is less than one year old; and
boosting the vaccination of the poultry animal when the poultry animal is between about 50 weeks old and about 60 weeks old, the boosting comprising administering killed virus if the poultry animal molted subsequent to the vaccination, and the boosting comprising administering live virus if the poultry animal did not molt subsequent to the vaccination.

2. The method as claimed in claim 1, wherein the killed virus includes at least one of an inactivated poliovirus (IPV) vaccine, a whole cell pertussis vaccine, a rabies vaccine, a hepatitis A virus vaccine, and a combination thereof.

3. The method as claimed in claim 1, wherein the live virus includes at least one of a varicella-zoster vaccine, oral poliovirus (OPV) vaccine, a yellow fever virus vaccine, and a combination thereof.

4. The method as claimed in claim 1, further comprising:
implementing a Salmonella enteritidis environmental monitoring program, which includes at least one testing protocol.

5. The method as claimed in claim 4, wherein the testing protocol comprises testing of chick papers at delivery.

6. The method as claimed in claim 4, wherein the testing protocol comprises performing an environmental test at about 14 weeks to about 16 weeks post molt.

7. The method as claimed in claim 4, wherein the testing protocol comprises performing an environmental test at about 40 weeks to about 45 weeks post molt.

8. The method as claimed in claim 4, wherein the testing protocol comprises performing an environmental test at about 4 weeks to about 6 weeks post molt.

9. A system for generating a Salmonella free egg, the system comprising:
a poultry animal that was vaccinated when the poultry animal was less than one year old;
the poultry animal having been boosted when the poultry animal was between about 50 weeks old and about 60 weeks old, the boosting comprising administering killed virus if the poultry animal molted subsequent to the vaccination, and the boosting comprising administering live virus if the poultry animal did not molt subsequent to the vaccination.

10. The system as claimed in claim 9, wherein the killed virus includes at least one of an inactivated poliovirus (IPV) vaccine, a whole cell pertussis vaccine, a rabies vaccine, a hepatitis A virus vaccine, and a combination thereof.

11. The system as claimed in claim 9, wherein the live virus includes at least one of a varicella-zoster vaccine, oral poliovirus (OPV) vaccine, a yellow fever virus vaccine, and a combination thereof.

12. The system as claimed in claim 9, wherein the poultry animal is monitored by a Salmonella enteritidis environmental monitoring program, which includes at least one testing protocol.

13. The system as claimed in claim 12, wherein the testing protocol comprises testing of chick papers at delivery.

14. The system as claimed in claim 12, wherein the testing protocol comprises performing an environmental test at about 14 weeks to about 16 weeks post molt.

15. The system as claimed in claim 12, wherein the testing protocol comprises performing an environmental test at about 40 weeks to about 45 weeks post molt.

16. The system as claimed in claim 12, wherein the testing protocol comprises performing an environmental test at about 4 weeks to about 6 weeks post molt.

17. A method of generating a Salmonella free egg, the method comprising:
administering a vaccination to a poultry animal that is less than one year old;
boosting the vaccination to the poultry animal when the poultry animal is between about 50 weeks old and about 60 weeks old, the boosting comprising administering killed virus if the poultry animal molted subsequent to the vaccination, and the boosting comprising administering live virus if the poultry animal did not molt subsequent to the vaccination, the administering live virus comprising providing live virus prior to molting and providing live virus again subsequent to molting; and
harvesting an egg that was laid by the poultry animal.

18. The method as claimed in claim 17, wherein the killed virus includes at least one of an inactivated poliovirus (IPV) vaccine, a whole cell pertussis vaccine, a rabies vaccine, a hepatitis A virus vaccine, and a combination thereof.

19. The method as claimed in claim 17, wherein the live virus includes at least one of a varicella-zoster vaccine, oral poliovirus (OPV) vaccine, a yellow fever virus vaccine, and a combination thereof.

20. The method as claimed in claim 17, further comprising:
implementing a Salmonella enteritidis environmental monitoring program, which includes at least one testing protocol.
